(19) United States
(12) Patent Application Publication
(10) Pub. No.: US 2026/0094266 A1
(43) Pub. Date: Apr. 2, 2026

(54) DIGITAL SYSTEM FOR CELL ASSAYS USING LABEL FREE MICROSCOPY

(71) Applicant: NANOLIVE SA, Tolochenaz (CH)

(72) Inventors: Mathieu FRECHIN, Marly (CH); Lorenzo ARCHETTI, Ecublens (CH)

(21) Appl. No.: 19/112,011

(22) PCT Filed: Sep. 13, 2023
(86) PCT No.: PCT/EP2023/075199

§ 371 Date: Mar. 14, 2025

(30) Foreign Application Priority Data
Sep. 15, 2022 (EP) 22195848.1
Mar. 20, 2023 (EP) 23162948.6

(51) Int. Cl. G06T 7/00; G01N 33/50; G06T 7/12; G06T 7/40; G06T 7/50

(52) U.S. Cl. CPC G06T 7/0012; G01N 33/5091; G06T 7/12; G06T 7/40; G06T 7/50; G06T 2207/10056; G06T 2207/10101; G06T 2207/10152; G06T 2207/20076; G06T 2207/20081; G06T 2207/30024

(57) ABSTRACT

A method of performing cell assays, comprising: inputting in a computing system a digital refractive index (RI) image of a sample containing a plurality of cells, applying a segmentation algorithm in said computing system to process said digital RI image configured to locate and define an outer boundary of each cell of said plurality of cells, applying in said computing system RI values from the RI image to the segmented cells, calculating in said computing system from the RI values, metrics including any of composition, structure and shape of each segmented cell, evaluating the metrics with a machine learning module in said computing system to classify a physiological state of each of the cells.

The present invention relates to a digital system for performing cell assays using images captured by label free microscopy techniques.

Multicellular organisms rely on a set of genetically encoded mechanisms for the elimination of damaged, harmful or superfluous cells[1,2]. Programmed cell death (PCD) is not restricted to multicellular organisms, in which it is key for homeostasis[3], it is also found in unicellular eukaryotes[4], and in some prokaryotes[5]. PCD is to be opposed to accidental cell death (ACD) resulting from the exposition of cells to severe chemical (e.g., extreme pH), physical (e.g., high pressure) or mechanical aggressions (e.g., shear forces). Implications are that PCD can be accelerated, delayed or stopped using drug-mediated or genetic perturbations of the key molecular pathways supporting it[6] and as such, PCD is the subject of numerous pharmacological research efforts. ACD however represents an uncontrollable type of cell death due to the irreversible destruction of essential biological structures. It is therefore of great interest for drug research to be able to identify which cells die or not upon drug treatment, as well as to detect the broad type of cell death occurring together with the dynamics of cell death progression. To do so, many technologies have been developed over the years.

Current solutions for quantifying cell death include:

### Label-Based (Fluorescent) Approaches Usable During Time Lapse Experiments

The basic principles are the same for all fluorescent live assays, a cell impermeant dye is placed in the medium where it is not fluorescent, when they die of necrosis, cells become permeable, the fluorescent compound penetrates the cell and the cell becomes fluorescent. It is usually using a variant which provides a green signal.

While necrosis is easily detected through the physical alteration of the cell membrane, apoptosis, which is a more refined and control process supposed to maintain the impermeability of the membrane, requires a molecular approach to be detected. Fluorescent conjugates of annexin V are used to identify apoptotic cells. The human vascular anticoagulant annexin V, a calcium dependent phospholipid-binding protein that has a high affinity for the anionic phospholipid phosphatidylserine (PS). In normal healthy cells, PS is located on the cytoplasmic face of the plasma membrane, pointing toward the inside of the cell, simply speaking. However, during apoptosis, the plasma membrane undergoes structural changes that include translocation of PS from the inner to the outer leaflet (extracellular side) of the plasma membrane, PS points towards the medium, it is exposed to the exterior and annexin V that is present in the medium can bind to it and becomes detectable because of a local concentration. Apoptosis is then detected by the apparition of red cells.

An obvious problem of those fluo-based assays is that the fluorescence excitation as well as the toxicity of the fluo compounds accelerate and sometimes even trigger the death of cells that would have not died otherwise. The technique alters the result.

### Label-Based Approaches Compatible Only With End Point Measurement

Those techniques are very popular, the most used cell death assays performed over the world, for few reasons, they are simple to analyze, affordable, and installed since cell biology exists. They require plate readers, scintillation counter or basic screening microscopes to be run and are easy to perform.

A first approach consists in loading cells with a specific compound such as Chromium 51 (or a fluorescence enhancing ligand) and to measure its release after cytolysis. It is broadly accepted that the measured signal correlates directly with the number of lysed cells, however an apoptotic cell may not leak material therefore such assay is fundamentally skewed. It can be found in all suppliers of biological research material.

Alternatives to the release assays described above rely on the measurement of population (non-) proliferation based on DNA synthesis measurement. To do so, BrdU—a modified base—is loaded on cells and later detected using a monoclonal antibody. It is an end-point measurement of global DNA synthesis, which is believed and, most importantly, accepted by the community, to be a good proxy for cell growth. A plate reader is most suited for such test.

The most efficient proliferation assay for a plate reader setup is the ATP detecting luminescence assay. The concept however requires cells to be lysed and therefore only end point measurements are possible. Once cells are lysed, a reactant, the D-luciferin, is added to the cell extract and the light produced by the mixture is proportional to the amount of ATP, which is accepted to be a good proxy for population size. This approach is suitable for large-scale screening in plate readers since it is the quickest and the cheapest of all the proliferation assays that are mentioned here.

The most precise cell death assay for a plate reader setup is the caspase 3/7 activation assay. The concept allows for the measurement of cell death in screening microscopes and plate readers. The fluorescence version allows for continuous, live monitoring of apoptosis, while the luminescence versions is older and requires multiple end-point measurements. The luminescence version relies on the release of caspase 3/7 upon apoptosis activation, this enzyme will modify and activate a luminescent compound. The fluorescent version relies on the late destruction of DNA upon apoptosis activation, the freed DNA will then interact with and activate a fluorescent compound that is normally not able to reach DNA because of the nuclear membrane.

### Label-Free Approaches

It is known to use a combination of a microscopy device for quantitative phase imaging or phase contrast and software to detect cell death, for instance determining cell size or doing cell counting as a proxy to evaluate cell death. Existing commercial label free systems are not actual assays and do not have an automated solution for cell death prediction. Therefore, they cannot predict if a cell dies and thus cannot predict if it is dying of necrosis or apoptosis.

Two studies were published about predicting the death of cells within label free images.

In a first publication of Verduijn et al. in 2021 [7], called “Deep learning with digital holographic microscopy discriminates apoptosis and necroptosis” and published in Cell Death Discovery, the images were acquired using digital holography, and the predictions of necrotic and apoptotic cells were done using deep learning.

In a second publication of Hu et al. in 2022 [8], called “Live-dead assay on unlabeled cells using phase imaging with computational specificity” and published in Nature Communications, the images were acquired using quantitative phase imaging and the predictions of dead versus live cells were done using deep learning.

Both the imaging approaches as well as the algorithmic solutions described in these publications are not adapted for automated cell death detection in a fully integrated environment.

### SUMMARY OF THE INVENTION

It is an object of the invention to provide a system and method of performing cell assays that allows to accurately predict the physiological state of a cell in a label-free manner.

It is advantageous to provide a system and method of performing cell assays in a label-free manner that is rapid.

It is advantageous to provide a system and method of performing cell assays in a label-free manner that is reliable.

It is advantageous to provide a system and method of performing cell assays in a label-free manner that is economical to produce, use and maintain.

It is advantageous to provide a system and method of performing cell assays in a label-free manner that is easy to use.

It is advantageous to provide a system and method of performing cell assays in a label-free manner that allows to accurately distinguish between the physiological states of alive or dead by apoptosis or dead by necrosis.

Disclosed herein is a method of performing cell assays, comprising:
inputting in a computing system a digital refractive index (RI) image of a sample containing a plurality of cells,
applying a segmentation algorithm in said computing system to process said digital RI image configured to locate and define an outer boundary of each cell of said plurality of cells,
applying in said computing system RI values from the RI image to the segmented cells to generate a mask image of the segmented cells with the same dimensions as the digital RI image of the cells prior to segmentation,
calculating in said computing system from the RI values, metrics including any of composition, structure and shape of each segmented cell,
evaluating the metrics with a machine learning module in said computing system to classify a physiological state of each of the cells selected from a group of physiological states including or consisting of alive, apoptotic and necrotic.

In an advantageous embodiment, the method comprises generating said RI image by an optical diffraction tomography microscope.

In an advantageous embodiment, the step of generating the RI image comprises: acquisition of a set of raw hologram images for a plural number of illumination angles, computation of the 3D distribution of RI from the set of acquired holograms in the computing system.

In an advantageous embodiment, the metrics includes a set of shape descriptors such as a form factor, a 2D or 3D geometrical shape, a volume, an area, a perimeter, a surface, an eccentricity, a solidity, a compactness, an orientation, a set of zernike shape features.

In an advantageous embodiment, the metrics further include any one or more of:
an intensity;
an intensity distribution;
a set of Haralick texture features such as a granularity;
a set of Tamura texture features
a dry mass;
an Euler number.

In an advantageous embodiment, the metrics further includes said intensity.

In an advantageous embodiment, the metrics further includes said intensity distribution.

In an advantageous embodiment, the metrics further includes said Haralick texture features.

In an advantageous embodiment, the step of classifying a physiological state of each of the cells comprises calculating a probability of each cell to be within one of a defined plurality of said physiological states.

In an advantageous embodiment, the method includes displaying on a screen an image of the cells illustrating the physiological state of the cells by applying a different color representing each physiological state.

Also disclosed herein is a system for performing cell assays including:
an optical diffraction tomography microscope and
a computing system configured to receive 3D RI data from said optical diffraction tomography microscope, the computing system comprising hardware including a microprocessor and a memory, and program modules installed and executable in the hardware, said program modules including:
a segmentation algorithm in to process said digital RI image configured to locate and define an outer boundary of each cell of said plurality of cells,
a program module applying said RI values from the RI image to the segmented cells to generate a mask image of the segmented cells with the same dimensions as the digital RI image of the cells prior to segmentation
a program module calculating from the RI values, metrics of each segmented cell,
a machine learning module evaluating the metrics to classify a physiological state of each of the cells selected from a group of physiological states including or consisting of alive, apoptotic and necrotic.

In an advantageous embodiment, the optical diffraction tomography microscope is configured to acquire a set of raw hologram images for a plural number of illumination angles, and the computing system is configured to compute a 3D distribution of RI from the set of acquired holograms.

In an advantageous embodiment, the program module calculating metrics from the RI values, is configured to calculate metrics including a set of shape descriptors such as a form factor, a 2D or 3D geometrical shape, a volume, an area, a perimeter, a surface, an eccentricity, a solidity, a compactness, an orientation, a set of zernike shape features.

In an advantageous embodiment, the program module calculating metrics from the RI values, is configured to calculate metrics including any one or more of:
an intensity;
an intensity distribution;
a set of Haralick texture features such as a granularity;
a set of Tamura texture features
a dry mass;
an Euler number.

In an advantageous embodiment, the metrics further includes said intensity.

In an advantageous embodiment, the metrics further includes said intensity distribution.

In an advantageous embodiment, the metrics further includes said Haralick texture features.

In an advantageous embodiment, the segmentation algorithm includes a succession of rule-based signal processing steps, including any one or more of the operations of blurring, sharpening, thresholding, propagation, object filtering, fusion and fission, signals addition, multiplication, subtraction, and division.

In an advantageous embodiment, the machine learning algorithm comprises or consists of an ensemble machine learning classifier.

The present invention relates to a computational tool designed to detect two types of death experienced by the cells imaged by an optical diffraction tomography microscope that incorporates diffraction tomography imaging devices. The computational tool is named herein the live cell death assay (LCDA) and can be triggered in one click by the user. The process is articulated in three fully automatized steps, without any input required from the user. Those three broad steps are the following: i) the cells recorded with the optical diffraction tomography microscope are segmented using a rule-based approach and cell segmentation masks are saved as images. ii) The segmented cells are processed to calculate the texture and intensity of the refractive signal within the limits of the segmentation made at the previous step, as well as to calculate the shape features of the segmented cells and finally iii) a machine learning model interprets the set of numbers characterizing each segmented cell to define which cells are alive, necrotic or apoptotic. The user can then analyze the provided metrics to determine over time how perturbing factors (drugs, mutations, cell type, environmental changes, mechanical cues etc.) trigger or prevent cell death.

### BRIEF DESCRIPTION OF THE FIGURES

FIG. 1: The device workflow—Description of the acquisition sequence corresponding to one temporal cycle at all locations of a sample vessel. For long-term imaging, an unlimited number of cycles can be acquired. The reconstruction step is displayed showing schematically the major processing steps from the acquired holograms set to the MIP map generation.

FIG. 2: The user workflow—The user prepares his cells of interest in wells or multi-wells plates and might choose to subject his cells with a perturbing factor (drugs, mutations, cell type, environmental changes, mechanical cues etc.). He then uses an optical diffraction tomography microscope to acquire videos of his cells, selects which of those videos to analyze and launches the live cell death assay in one click. Then, the live cell death assay automatically segments, classify and quantify the cells to provide to the user cell shape, texture and intensity metrics as well as each cell classes and related probabilities.

FIG. 3: Schematic of the technical data workflow of the live cell death assay (LCDA)—Cells in images obtained with the optical diffraction tomography microscope are segmented using a rule-based approach. A set of a plurality of metrics, possibly up to hundreds of metrics, are then calculated for each segmented cell and these cell's signatures are then provided to a predefined machine learning model whose role is to attribute a probability of being alive, dead by apoptosis or dead by necrosis. The highest probability class becomes the cell's state. Cell's metrics and classes can then be graphically represented.

FIGS. 4a to 4e: Overview of the metrics extracted from each single cell segmented in the segmentation mask. In FIG. 4a, showing the general overview, from left to right are represented the nature of the extracted metrics by order of importance. FIGS. 4b to 4e illustrate in more detail various metrics of the general overview of FIG. 4a. Shape metrics (FIG. 4b) are the most essential. The shape metrics allows the LCDA to perform, albeit with non-ideal performances, but to perform nonetheless, although it is preferred to include the addition of at least intensity features (FIG. 4d). It is further preferable to include intensity distributions features (FIG. 4c). Further advantageous metrics include texture features, in particular Haralick texture features. The various aforementioned preferred and advantageous metrics increases the sharpness of the LCDA an ensemble machine learning classifier predictions.

FIG. 5: Overview of segmentation masks produced by LCDA—The LCDA provides the possibility to represent the cell's classification probabilities—alive, apoptotic, necrotic—as colored masks overlays.

FIG. 6: Benchmarking of the LCDA with the Fluorescence cell death assay kit—We imaged 3t3—derived preadipocytes on an optical diffraction tomography combining RI recording and epifluorescence, with and without addition of camptothecin at 5 uM, a proapoptotic drug. The comparison of the live cell death assay classes,—namely alive, necrotic and apoptotic—(image line A) with the cell's status obtained from state-of-the-art, live fluorescence cell death assay (image lines B, C, D showing respectively the merge of the green and red fluorescent signals, the red, apoptotic specific Annexin V signal and finally the green, dominant, nuclear signal for necrosis). This experiment shows that the live cell death assay (LCDA) performs as well as its fluorescent counterpart (E and F) with the removed burden of fluorescent sample preparation and photo-plus cytotoxicity brought by the chemical fluorescent compounds, known to accelerate and even trigger cell death.

FIG. 7: Application of LCDA to quantify Ebastine-induced cell death—HeLa cells grown in two wells were used as (A) control and (B) treatment experiment. The later consist in treating cells right before acquiring them with 50 uM of Ebastin, a compound known to induce cell death. This figure shows that cells are automatically segmented, quantified and classified as living or dead cells within the LCDA, and represented with colored overlays and that their metrics can be plotted directly in the environment. Here (C) the plotted number of dead cells predicted over time in control and ebastin treated cells shows a higher cell death rate in the treated condition already at 5 h post treatment.

FIG. 8: List of the live cell death assay—specific metrics—The metrics produced by the live cell death assay (LCDA) can be organized in four groups. Firstly, the LCDA predictions, are cells alive, apoptotic or necrotic, as well as the respective probability related to those states. Secondly, the global counts, describing in general how many cells are dead or alive, in relative or absolute terms. Thirdly, dynamic status that are linked to the evolution of the probability of cells to die or to stay alive (1-probability to die) over time, cells are stressed if the probability of dying increases over time, and are recovering if they probability of being dead decreases over time. Fourthly and finally, specific counts, being the relative and absolute number of apoptotic or necrotic cells.

### DETAILED DESCRIPTION OF EMBODIMENTS AND EXAMPLES

### Optical Diffraction Tomography Detection

A mammalian cell that is probed by an optical field leaves a footprint in the complex phase of the beam that is representative of it's the integrated refractive index (RI) distribution along the beam propagation direction. A holographic detection of this optical field enables the recovery of this phase which however does not allow for an unequivocal retrieval of the RI distribution. Optical diffraction tomography (ODT) combines such a holographic detection of the complex field with a scanning apparatus able to create a plurality of illumination angles. This combination allows to reconstruct unequivocally the 3D RI distribution within a sample with a resolution up to roughly twice as large as the one that we can achieve with the same imaging objective with classic widefield imaging techniques. In order to retrieve this 3D structure, one must collect optical phases maps from multiple directions equally distributed around the optical axis of the objective.

To obtain this 3D RI structure that characterizes a living cells sample, a sequence of steps is carried out in the microscope that is used in this invention and whose design is patented. Once the vessel containing the living cells under analysis is placed onto the incubated stage of the microscope and after a thermalization time, the user can launch the acquisition procedure which consists in the three main following steps:
Calibration of the probing beam and the reference beam in order to create the optimum conditions for the holographic detection of the optical phase signal at each illumination angle of the rotational scanning
Acquisition of a set of raw images (holograms) for a sufficient number of illumination angles (preferably at least 20, and ideally more than 50)
Computation of the 3D distribution of RI from the set of acquired holograms

### Calibration Step

First of all, the camera signal is used to center the Class I laser beam that probes the sample onto the sensor for all illumination directions that the rotational scanning will generate. A second step consists of adjusting the reference beam, in order to generate holograms during the rotational scanning whose properties will be suitable for accurate phase computation.

### Acquisition Step

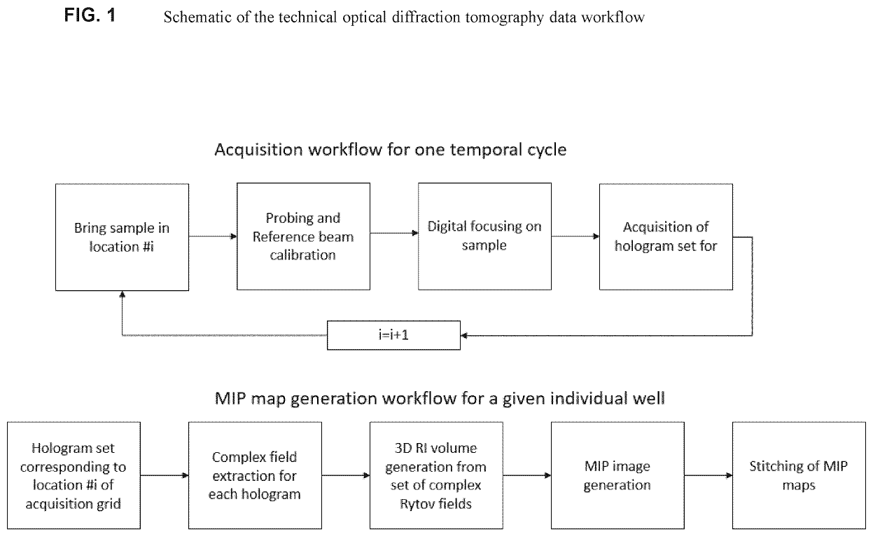

Once the microscope is properly calibrated and thus adjusted to the sample and environmental conditions, a step of digital autofocus is carried out to place the sample at the focal plane of the microscope objective, and a sequence of holograms is further acquired during a rotational scanning at a given chosen location of the sample vessel. It is possible to run multiple acquisitions in adjacent locations to reconstruct 3D volumes of an extended field of view. It is possible, especially for benchmarking purpose, to acquire epifluorescence images with up to 3 channels of excitation light in sequence. Once the acquisition sequence is completed at a given location, the automated stage of the ODT microscope is bringing the sample to another location (e.g. in another well of a multiwell plate where different controlled conditions are set by the user, FIG. 1). A calibration check step is carried out to ensure that optimal holographic conditions are met in this new well taking into account potential opto-mechanical discrepancies. A cycle of acquisition is completed when holograms have been acquired in all locations of the sample vessel. The optical diffraction tomography technique being label free and not invasive, it is enabling long term imaging of living samples over days and thus the acquisition of an unlimited number of cycles.

### 3D Reconstruction Step

The reconstruction step relies on an algorithm that delivers 3D RI data when fed with a set of holograms obtained following the aforementioned method. To reduce the amount of data, a maximum intensity projection (MIP) map is generated at each location of the sample vessel where sets of holograms have been acquired from a subset of the 3D volume (FIG. 1). When acquisition sequences are run in adjacent locations of a rectangular grid, the MIP maps are stitched together to form an extended filed of view (FoV) of the sample while keeping the original high lateral resolution, specific of optical diffraction tomography.

Those MIP maps represent the input data on which the Live Cell Death Assay is running.

### Live Cell Death Assay Analysis

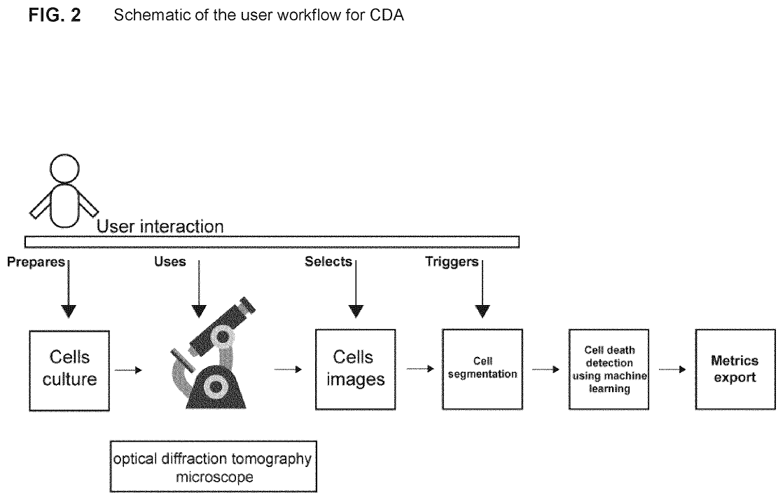

The last part of the user workflow (FIG. 2) comprises triggering the live cell death assay analysis.

Cell death displays morphological alterations that can be captured by a diffraction tomography microscope. Firstly, Apoptosis, the major form of PCD, exhibits plasma membrane blebbing, cytoplasmic shrinkage, pyknosis, karyorrhexis, and culminates with the formation of apparently intact small vesicles, or apoptotic bodies, which are cleaned up in healthy organisms by cells with phagocytic activity. Secondly, necrosis, the major form of ACD, shows no features of apoptosis and terminates with the disposal of cells [10]. These two major types of cell death are very stereotypic and easily distinguishable from one another within optical diffraction tomography microscopic images. Here is how the live cell death assay do such task.

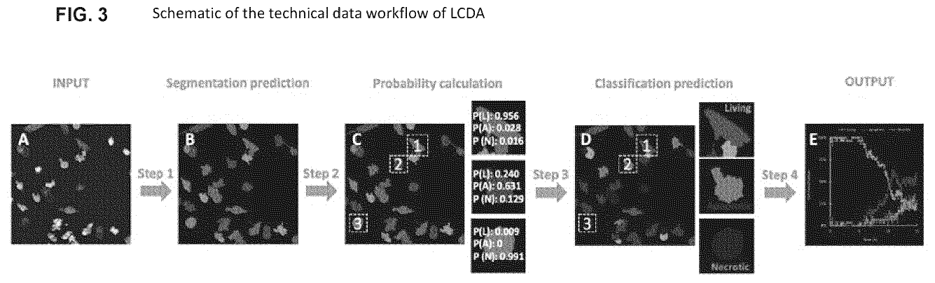

Firstly, the cells contained in the image files (or MIP maps) are segmented using a succession of rule-based signal processing steps (FIG. 3). This occurs in a segmentation algorithm that includes a succession of rule-based signal processing steps targeting bio-specific signal characteristics. It may be noted that the succession of rule-based signal processing steps targeting bio-specific signal characteristics is different to a basic refractive index thresholding that cannot be considered as a segmentation algorithm as it does not take into consideration bio-specific elements and as such is not generalizable. In embodiments of the present invention, the rule-based signal processing steps include any one or more of the operations of blurring, sharpening, thresholding, propagation, object filtering, fusion and fission, signals addition, multiplication, subtraction, and division.

The algorithm may advantageously comprise the following steps:
i) a cellular “core”, or primary object, that indicates the approximative center of each cell, is identified. In the absence of a simple cell center indicator such as Hoechst in fluorescence microscopy, the algorithm according to an embodiment works towards transforming the typical cellular center signals, structures, and textures into a single central cellular object. To do so, we specifically threshold the image with a maximum threshold to remove objects such as lipid droplets or nucleoli that are of too strong signals, we then blur the image using a gaussian filter, and fill signal holes with size specific feature detectors. This allows to generate a signal gradient, fainter at the edge of cells and stronger at the center of cells that can then be used to identify primary objects using a simple thresholding step.
ii) This primary object, or cellular “core” is then used as the starting point to detect the precise boundaries of cells and create a secondary object, this secondary object is our cellular mask. To do so, a propagation algorithm is used to propagate the primary object into a succession of mildly to strongly blurred raw refractive index signal. The propagation algorithm itself assesses in an iterative manner if the next increment of the secondary object faces a strong signal gradient, typical of the end of the plasma membrane of the cell that needs to be segmented. If it does the propagation is terminated. By propagating in multiple blurred images, the algorithm assesses if the propagation tends to stop because of a local texture or object maximum or because it has found true boundaries.

Those boundaries are the limits that define precisely what segmented cells are; what belongs to each cell and what is external to it. The segmented cells are stored as a mask image that possesses the same dimensions as the RI images such that with a simple overlay of the mask image over the RI image, the proper refractive index signal can be attributed to the proper cell.

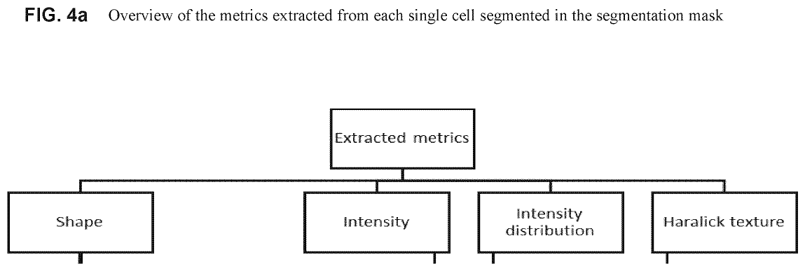

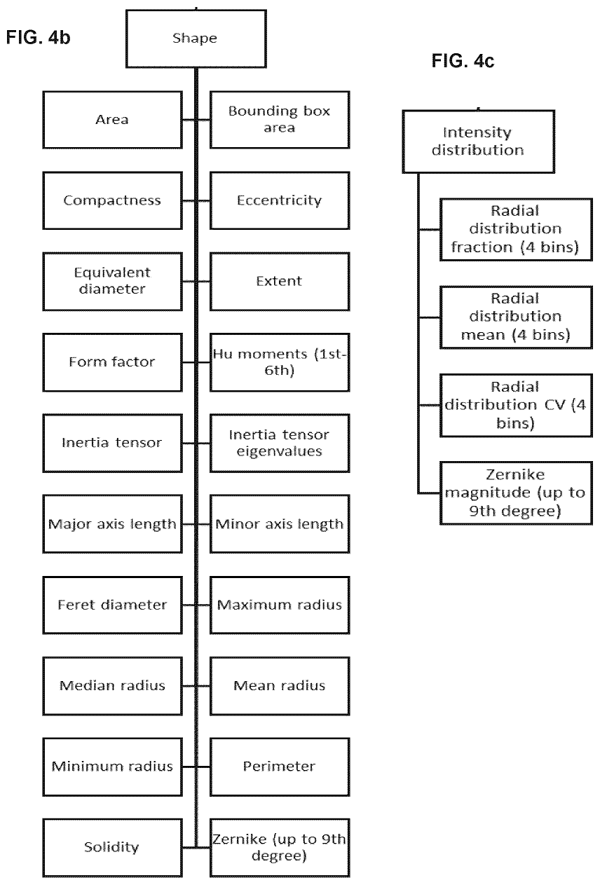

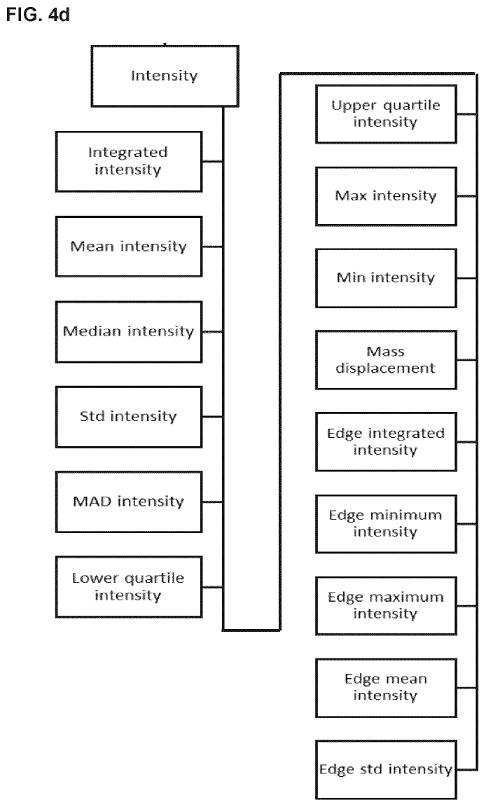

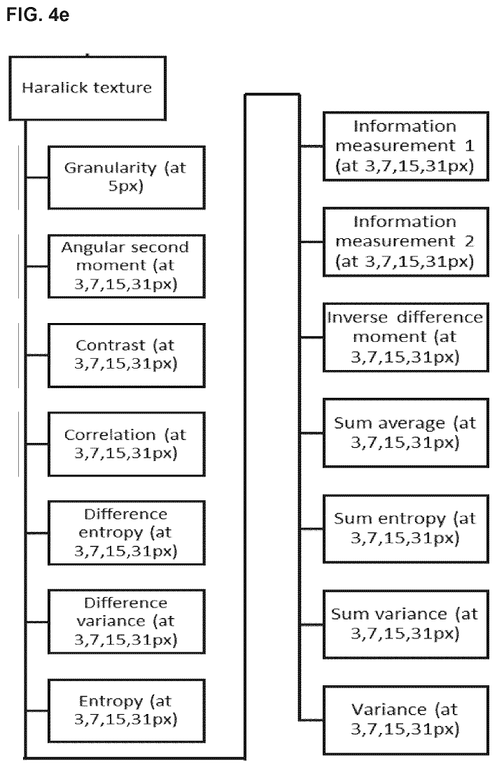

Secondly, the segmented cells are used in combination with their respective refractive index signal with a programmed algorithm to calculate the texture metrics of the refractive index signal contained within each cell. Such textures metrics are meant to capture the organellar organization of the cell. They are also used to calculate the intensity metrics of each cell, which is related to how much dry mass each cell contains. They are finally used to calculate the shape descriptors that characterize how large, small or complex the shape of a cell is (see FIGS. 4a to 4e). Altogether, those metrics carry in a complex way the physiological status of a cell and its probability to live or die and if they might die, which process is likely to occur, apoptosis or necrosis (FIG. 3).

To capture the information carried out by the cellular metrics extracted at the previous step, which are out of reach for the human mind due to the large dimensionality of the data, but also unoptimized for computer analysis due to unknown collinearities and large differences in signal dynamics, the next step consists in normalizing the data and apply dimensional reduction using principal component analysis. After this step, each cell segmented within the refractive index signal images acquired by the optical diffraction tomography microscope possesses a numerical signature composed of the many metrics calculated before (see FIGS. 4a to 4e). This signature is evaluated by a machine learning model that will attribute a score for the cell to be alive, apoptotic or necrotic (see FIG. 3). The machine learning model is created and trained outside of the live cell death assay in a dedicated environment designed for the creation of machine learning model. The live cell death assay is not about training machine learning models, it is about applying it to classify cells.

In preferred embodiments of the invention, the used machine learning model is an ensemble machine learning algorithm because of its capacity to capture non-linear decision boundaries and to generalize well. Such algorithm was developed in response to the fact that a single decision tree is overly sensitive to training data. The many trees that constitute an ensemble machine learning classifier each generate a vote (a local prediction) to make the final prediction. This helps avoiding overfitting, because each of the many trees is built on a random subset of the training data (using bootstrap replica to generate by random repetition a sampling set as large as the training data) and a random subset of explanatory variables. In a nutshell, these algorithmic elements make the ensemble machine learning classifier less sensitive to training data and cause less variance between trees predictions.

Those peculiarities are essential when classifying biological states: in biology, because they are very long and complex to produce, the samples one can have access to in the duration of a development process are necessarily, whether it is through direct production or collaborations, a narrow representation of the possible diversity of the data the classifier might be confronted in users'laboratories all over the world. In fact, in biology it is arguably impossible to acquire training dataset that are representative of the possible data the classifier will be exposed to, and the need to mitigate this fact imposes an ensemble machine learning classifier.

The probabilities of each cell to be alive, necrotic or apoptotic are then evaluated and each cell is attributed the state with the highest probability. There can only be one class per cell as all these states are biologically exclusive. A cell cannot be alive and dead at the same time, or apoptotic and necrotic at the same time.

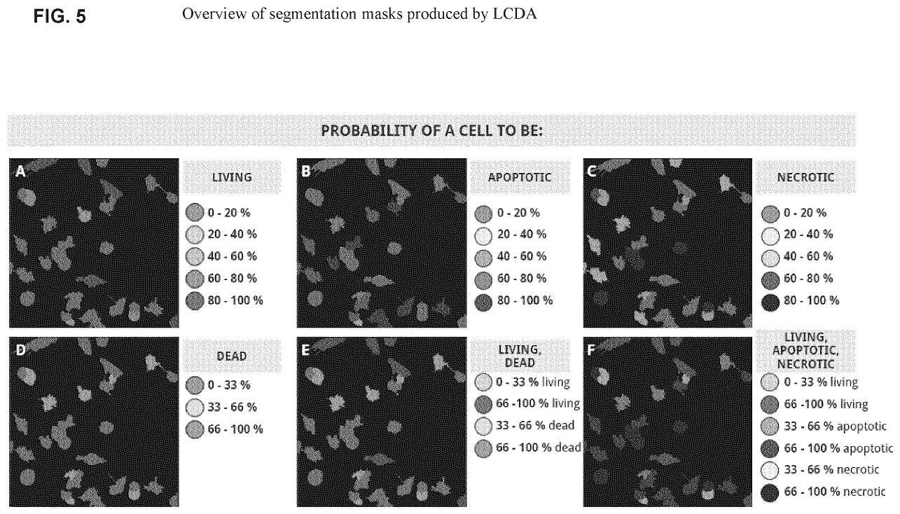

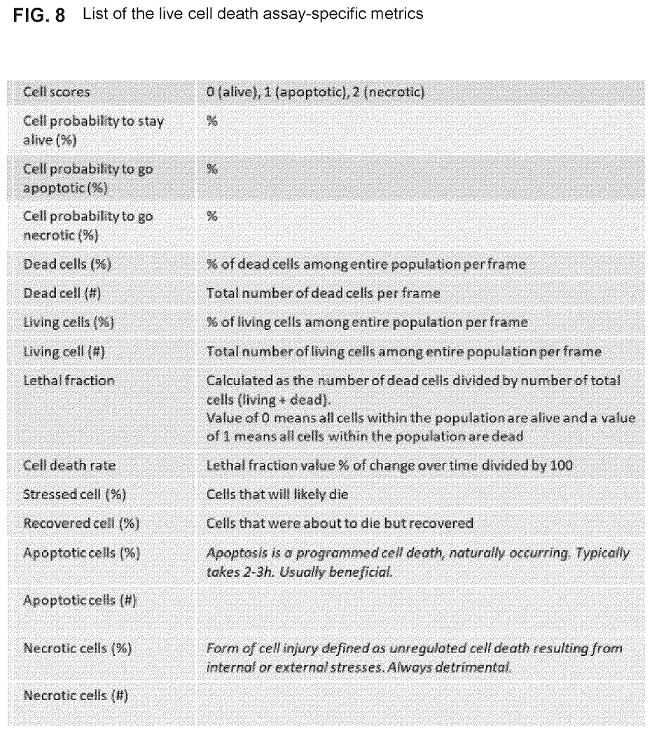

The metrics derived from this classification step that are listed within the table in FIG. 8 are then calculated in a last step and provided to the user for subsequent analysis, they can be plotted as we will see below, or represented as image overlays (FIG. 5).

A major effort of the cell death assay development is to benchmark the live cell death assay (LCDA) against state-of-the-art fluorescent live cell imaging assays designed to detect cell death, to assess at the same time the precision, effectiveness and practicality of the LCDA. The basic principles used by popular fluorescent assays in order to discriminate between living and dead cells are similar: a cell impermeable marker is placed in the medium, where it is not fluorescent; when cells die, their membrane becomes permeable and the compound is then able to penetrate inside the cells, binding to cellular structures (such as the DNA) and becoming fluorescent[11].

This type of necrotic cell death is easily detected through the physical alteration of the cell membrane and represents the largest amount of used cell death assays. However, apoptosis—which is a more refined and controlled process, maintaining the impermeability of the membrane—requires a molecular approach to be detected.

Fluorescent conjugates of annexin V are the favored way for identifying apoptotic cells[12]. Annexin V is a calcium dependent phospholipid-binding protein that has a high affinity for the anionic phospholipid phosphatidylserine (PS) [13]. In normal healthy cells, PS is exclusively located on the inner (cytoplasmic) side of the plasma membrane; however, during apoptosis, the plasma membrane undergoes structural changes that include the translocation of PS from the inner to the outer (extracellular) leaflet of the plasma membrane. As such, it is exposed to the annexin V present in the medium, which can bind to it and become detectable [12] by a phenomenon of local accumulation.

But an annexin V marker is not sufficient to distinguish between apoptotic and necrotic cells. The reason is the following: the membrane integrity of a necrotic cell is lost, annexin V can penetrate inside the necrotic cell, and from the inside bind PS, giving rise to a strong annexin V fluorescent signal while PS was not externalized.

To distinguish between apoptosis and necrosis it is necessary to use annexin V together with a necrosis marker. In this case, when only annexin V signal is detected, we can be sure that a cell is undergoing apoptosis, while when both signals are observed a cell is necrotic.

It is important to note that in in vitro studies, cells that undergo apoptosis will—at a certain point—inevitably become necrotic because ex vivo apoptosis is not followed by the complex phagocytic clearance mechanism leading to their disposal [14].

Typically, fluorescence-based cell death assays also include a living cell marker, in order to visualize living cells, which would otherwise remain undetected. Quantifying the amount of living cells is of critical importance since it would be otherwise impossible to normalize cell death metrics to the total population. But this is only true in fluorescence-only approaches that rely on fluorescence to segment all cells, while this benchmarking experiment relies on the segmentation of all cells based on the refractive index contained within the live cell death assay in order to reduce the amount of phototoxicity and cytotoxicity brought by fluorescent signals.

The main limitation of fluorescence-based assays—apart from increased time and reagent cost with respect to label-free techniques—is that the fluorescence excitation as well as the inherent toxicity of the fluorescent compounds accelerate and sometimes ever trigger the death of cells that would not have died otherwise, altering the results [15].

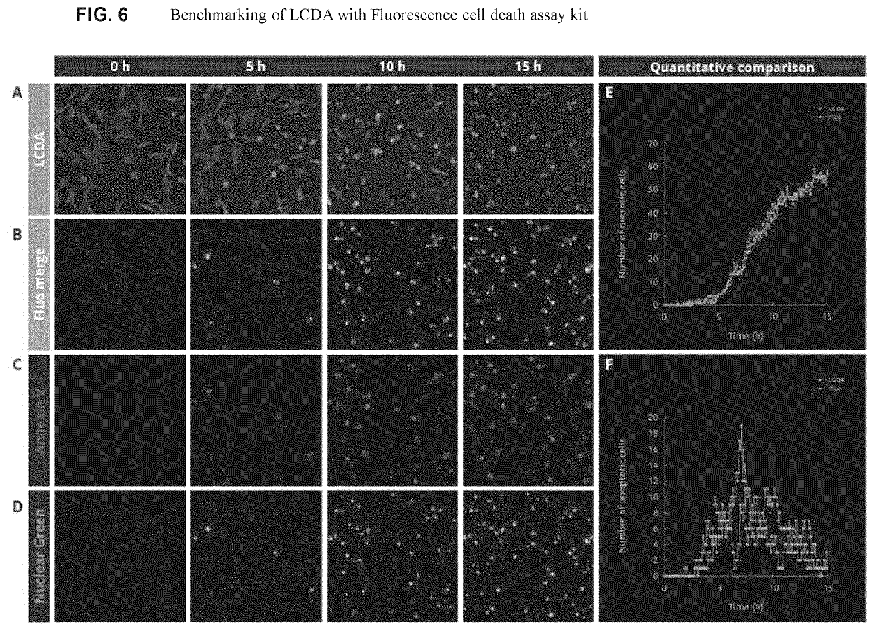

To validate the performance of the LCDA, the invention method was benchmarked with a state-of-the-art live fluorescent kit (FIG. 6). 3t3-derived preadipocytes were imaged on an optical diffraction tomography system combining RI recording and epifluorescence, with and without addition of camptothecin, a proapoptotic drug (FIG. 6). Annexin V-Cy5 and Nuclear Green DCS1 were used as fluorescent markers to detect apoptosis and necrosis, respectively. The RI images of the kit-stained cells were also analyzed with the LCDA. The cell death information provided by the fluorescent signals contained within the RI segmentation masks was then compared, with the LCDA results (FIG. 6, E and F).

3T3-L1-derived preadipocyte cells were seeded in a 35 mm dish at ˜30,000 cells/ml and let to grow for 48 h in DMEM supplemented with 10% FBS. 4 hours before starting the acquisition, the cells were treated with 2.5 μM camptothecin (abcam—ab120115) to induce apoptosis. 10 minutes before starting the acquisition, Annexin V-Cy5 and Nuclear Green DCS1 (abcam—ab14147, ab138905) were added to the medium, following the supplier's protocol. The dish was then imaged for 17.5 hours at a frequency of 1 image every 5 minutes for RI images and 1 image every 10 minutes for fluorescent images. Fluorescent images were captured in the FITC and Cy5 channels with the minimum intensity and exposure sufficient to obtain high quality images.

RI images were then analyzed and plotted with the LCDA module's features. Fluorescence was quantified by measuring the mean intensity in each cell using RI segmentations using an independent image analysis tool. Following the kit's recommendations, cells positive for Nuclear Green or Annexin V or both are considered dead. Cells positive for Nuclear Green (Annexin V positive or not) are necrotic, cells positive for Annexin V and negative for Nuclear Green are apoptotic.

Visually and by quantification, one observes that the fluorescent assay directly induces cell death, as early as 8 h after starting the acquisition. The addition of camptothecin accelerates greatly the death of cells and results in the whole cell population dying by the end of the experiment (FIG. 6).

The analysis was focused on the condition with camptothecin, which shows a typical response of cell death upon the addition of drug and a larger lethal fraction delta from start to finish.

The CDA captures similar cell death dynamics to the fluorescent assay. Focusing on specific cells, it is apparent that apoptosis is detected earlier by the CDA than by the fluorescent assay, while necrosis detection coincides. Quantifying the number of apoptotic and necrotic cells confirms that the CDA detects necrosis similarly to the fluorescent assay, while detecting apoptosis earlier and more reliably. It has been reported that PS detection only appears after cells have undergone clear, apoptosis-specific morphological changes. Notably, it has been observed that cytoskeleton rearrangements and depolymerization arise in the earliest phases of apoptosis[16], explaining why the LCDA is able to detect apoptosis earlier. Moreover, it has also been shown that PS externalization is not an essential component of the apoptotic phenotype[17] and can also appear in non-apoptotic cells[18].

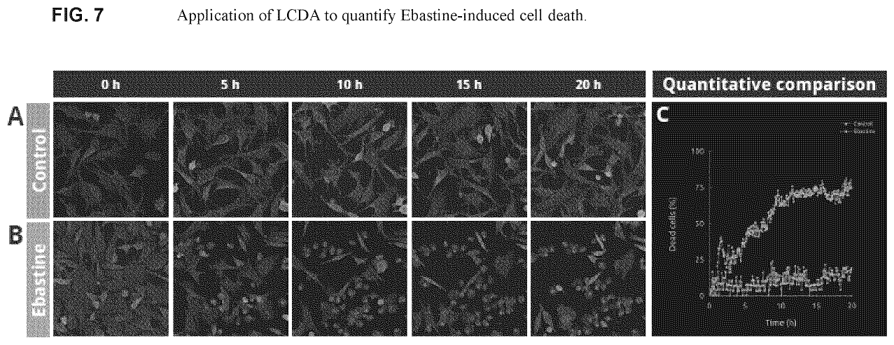

It was finally concluded by quantifying the capacity of another drug to kill cells, Ebastin, to validate the simplest of the uses of the LCDA. 3T3-L1-derived preadipocyte cells were seeded in 35 mm dish at ˜30,000 cells/ml and let to grow for 48 h in DMEM supplemented with 10% FBS. 1 hour before starting the acquisition, the cells were treated with 20 μM Ebastin (sigma—E9531) to induce necrosis and showed that untreated cells do not die while treated cells show a very clear curve of induced cell death (FIG. 7).

### LITERATURE REFERENCES

1. Conrad M, Angeli JPF, Vandenabeele P, Stockwell BR. Regulated necrosis: disease relevance and therapeutic opportunities. Nat Rev Drug Discov. 2016;15: 348-366. doi:10.1038/nrd.2015.6
2. Fuchs Y, Steller H. Live to die another way: modes of programmed cell death and the signals emanating from dying cells. Nat Rev Mol Cell Biol. 2015;16: 329-344. doi:10.1038/nrm3999
3. Fuchs Y. Programmed Cell Death in Animal Development and Disease.: 17.
4. Eisenberg T, Büttner S, Kroemer G, Madeo F. The mitochondrial pathway in yeast apoptosis. Apoptosis. 2007;12: 1011-1023. doi:10.1007/s10495-007-0758-0
5. Green DR, Fitzgerald P. Just So Stories about the Evolution of Apoptosis. Curr Biol. 2016;26: R620-R627. doi:10.1016/j. cub.2016.05.023
6. Galluzzi L, Bravo-San Pedro JM, Kepp O, Kroemer G. Regulated cell death and adaptive stress responses. Cell Mol Life Sci. 2016;73: 2405-2410. doi:10.1007/s00018-016-2209-y
7. Verduijn J. Deep learning with digital holographic microscopy discriminates apoptosis and necroptosis. Cell Death Discov. 2021; 10.
8. Hu C. Live-dead assay on unlabeled cells using phase imaging with computational specificity.: 8.
9. Farhat G, Mariampillai LD, Yang VXD, Czarnota GJ, Kolios M, CI US. (54) COMPUTING DEVICE AND METHOD FOR DETECTING CELL, DEATH IN A BIOLOGICAL SAMPLE.: 21.
10. Galluzzi L, Maiuri MC, Vitale I, Zischka H, Castedo M, Zitvogel L, et al. Cell death modalities: classification and pathophysiological implications. Cell Death Differ. 2007;14: 1237-1243. doi:10.1038/sj.cdd.4402148
11. Sasaki DT, Dumas SE, Engleman EG. Discrimination of viable and non-viable cells using propidium iodide in two color immunofluorescence. Cytometry. 1987;8: 413-420. doi:10.1002/cyto.990080411
12. van Engeland M, Nieland LJW, Ramaekers FCS, Schutte B, Reutelingsperger CPM. Annexin V-Affinity assay: A review on an apoptosis detection system based on phosphatidylserine exposure. Cytometry. 1998;31: 1-9. doi:10.1002/(SICI)1097-0320(19980101)31:1<1::AID-CYTO1>3.0.CO;2-R
13. Thiagarajan P, Tait JF. Binding of annexin V/placental anticoagulant protein I to platelets. Evidence for phosphatidylserine exposure in the procoagulant response of activated platelets. J Biol Chem. 1990;265: 17420-17423. doi:10.1016/S0021-9258(18)38177-8
14. Silva MT. Secondary necrosis: The natural outcome of the complete apoptotic program. FEBS Lett. 2010;584: 4491-4499. doi:10.1016/j. febslet.2010.10.046
15. Kiepas A, Voorand E, Mubaid F, Siegel PM, Brown CM. Optimizing live-cell fluorescence imaging conditions to minimize phototoxicity. J Cell Sci. 2020;133: jcs242834. doi:10.1242/jcs.242834
16. Ndozangue-Touriguine O, Hamelin J, Bréard J. Cytoskeleton and apoptosis. Biochem Pharmacol. 2008;76: 11-18. doi:10.1016/j.bcp.2008.03.016
17. Fadeel B, Gleiss B, Hogstrand K, Chandra J, Wiedmer T, Sims PJ, et al. Phosphatidylserine Exposure during Apoptosis Is a Cell-Type-Specific Event and Does Not Correlate with Plasma Membrane Phospholipid Scramblase Expression. Biochem Biophys Res Commun. 1999;266: 8.
18. Shlomovitz I, Speir M, Gerlic M. Flipping the dogma—phosphatidylserine in non-apoptotic cell death. Cell Commun Signal. 2019;17: 139. doi:10.1186/s12964-019-0437-0

### LIST OF FEATURES

### System for Cell Assays

### Microscope

Optical diffraction tomography microscope
Control system
Rotating beam system
Sensor
   Computing system

RI image processing module

Cell assay module
Cell segmentation module
Cell RI image processing module
Machine learning module

User interface
Display
Input elements

1. A method of performing cell assays, comprising:
inputting in a computing system a digital refractive index (RI) image of a sample containing a plurality of cells,
applying a segmentation algorithm in said computing system to process said digital RI image configured to locate and define an outer boundary of each cell of said plurality of cells,
applying in said computing system RI values from the RI image to the segmented cells to generate a mask image of the segmented cells with the same dimensions as the digital RI image of the cells prior to segmentation,
calculating in said computing system from the RI values, metrics including any of composition, structure and shape of each segmented cell,
evaluating the metrics with a machine learning module in said computing system to classify a physiological state of each of the cells selected from a group of physiological states including or consisting of alive, apoptotic and necrotic.

2. The method of claim 1 comprising: generating said RI image by an optical diffraction tomography microscope.

3. The method of claim 2 wherein the step of generating the RI image comprises:
acquisition of a set of raw hologram images for a plural number of illumination angles,
computation of the 3D distribution of RI from the set of acquired holograms in the computing system.

4. The method of claim 1 wherein the metrics includes a set of shape descriptors such as a form factor, a 2D or 3D geometrical shape, a volume, an area, a perimeter, a surface, an eccentricity, a solidity, a compactness, an orientation, a set of zernike shape features.

5. The method of claim 4 wherein the metrics further include any one or more of:
an intensity;
an intensity distribution;
a set of Haralick texture features such as a granularity;
a set of Tamura texture features
a dry mass;
an Euler number.

6. The method of claim 5 wherein the metrics further includes said intensity.

7. The method of claim 6 wherein the metrics further includes said intensity distribution.

8. The method of claim 7 wherein the metrics further includes said Haralick texture features.

9. The method of claim 1 wherein the segmentation algorithm includes a succession of rule-based signal processing steps, including any one or more of the operations of blurring, sharpening, thresholding, propagation, object filtering, fusion and fission, signals addition, multiplication, subtraction, and division,

10. The method of any claim 1 wherein the step of classifying a physiological state of each of the cells comprises calculating a probability of each cell to be within one of a defined plurality of said physiological states.

11. The method of claim 1 wherein the machine learning algorithm comprises an ensemble machine learning classifier.

12. The method of claim 1 comprising displaying on a screen an image of the cells illustrating the physiological state of the cells by applying a different color representing each physiological state.

13. A system for performing cell assays including:
an optical diffraction tomography microscope and
a computing system configured to receive 3D RI data from said optical diffraction tomography microscope, the computing system comprising hardware including a microprocessor and a memory, and program modules installed and executable in the hardware, said program modules including:
a segmentation algorithm in to process said digital RI image configured to locate and define an outer boundary of each cell of said plurality of cells,
a program module applying said RI values from the RI image to the segmented cells to generate a mask image of the segmented cells with the same dimensions as the digital RI image of the cells prior to segmentation
a program module calculating from the RI values, metrics of each segmented cell,
a machine learning module evaluating the metrics to classify a physiological state of each of the cells selected from a group of physiological states including or consisting of alive, apoptotic and necrotic.

14. The system of claim 13 wherein optical diffraction tomography microscope is configured to acquire a set of raw hologram images for a plural number of illumination angles, and the computing system is configured to compute a 3D distribution of RI from the set of acquired holograms.

15. The system of claim 1 wherein the program module calculating metrics from the RI values, is configured to calculate metrics including a set of shape descriptors such as a form factor, a 2D or 3D geometrical shape, a volume, an area, a perimeter, a surface, an eccentricity, a solidity, a compactness, an orientation, a set of zernike shape features.

16. The system of claim 1 wherein the program module calculating metrics from the RI values, is configured to calculate metrics further including any one or more of:
an intensity;
an intensity distribution;
a set of Haralick texture features such as a granularity;
a set of Tamura texture features
a dry mass;
an Euler number.

17. The system of claim 16 wherein the metrics further includes said intensity.

18. The system of claim 17 wherein the metrics further includes said intensity distribution.

19. The system of claim 18 wherein the metrics further includes said Haralick texture features.

20. The system of claim 1 wherein the segmentation algorithm includes a succession of rule-based signal processing steps, including any one or more of the operations of blurring, sharpening, thresholding, propagation, object filtering, fusion and fission, signals addition, multiplication, subtraction, and division.

21. The system of claim 1 wherein the machine learning algorithm comprises or consists of an ensemble machine learning classifier.
